(19) United States
(12) Patent Application Publication
(10) Pub. No.: US 2013/0012714 A1
(43) Pub. Date: Jan. 10, 2013

(54) NOVEL SOLVATE OF DEXLANSOPRAZOLE

(75) Inventors: Buchi Reddy Reguri, Chennai (IN); Sampath kumar Upparapalli, Chennai (IN); Thirumurugan Kunchithapatham, Chennai (IN); Sabarinathan Natarajan, Chennai (IN); Thiyagarajan Sambashivam, Chennai (IN); Suresh Munuswamy, Chennai (IN); Susi Swaminathan, Chennai (IN)

(73) Assignee: ORCHID CHEMICALS & PHARMACEUTICALS LIMITED, Chennai, Tamil Nadu (IN)

(21) Appl. No.: 13/578,078

(22) PCT Filed: Feb. 4, 2011
(86) PCT No.: PCT/IB2011/050488

§ 371 Date: Aug. 30, 2012

(30) Foreign Application Priority Data
Feb. 11, 2010 (IN) 349/CHE/2010
May 5, 2010 (IN) 1258/CHE/2010

(51) Int. Cl. C07D 401/12

(52) U.S. Cl. 546/273.7

(57) ABSTRACT

The present invention provides novel solvate of dexlansoprazole (or R-lansoprazole), particularly diol solvate and its hydrate form of dexlansoprazole (or R-lansoprazole) which is chemically known as (R)-2-[[[3-methyl-4-(2,2,2-trifluoroethoxy)-2-pyridinyl]methyl]sulfinyl]-1H-benzimidazole compound of the formula (I), as well as processes for the preparation thereof. The said solvates are useful in the preparation of dexlansoprazole with enhanced chiral purity and better HPLC purity with less sulphone impurity content.

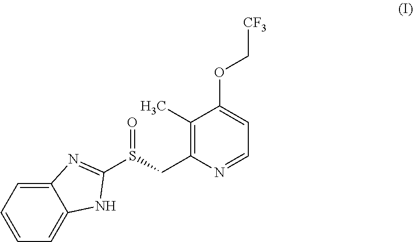

### FIELD OF THE INVENTION

The present invention provides novel solvate of dexlansoprazole (or R-lansoprazole), particularly diol solvate and its hydrate form of dexlansoprazole (or R-lansoprazole) which is chemically known as (R)-2-[[[3-methyl-4-(2,2,2-trifluoroethoxy)-2-pyridinyl]methyl]sulfinyl]-1H-benzimidazole compound of the formula (I), as well as processes for the preparation thereof. The said solvates are useful in the preparation of dexlansoprazole with enhanced chiral purity and better HPLC purity with less sulphone impurity content.

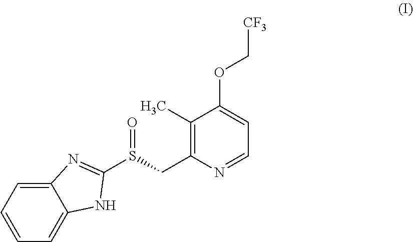

### BACKGROUND OF THE INVENTION

(R)-2-[[[3-methyl-4-(2,2,2-trifluoroethoxy)-2-pyridinyl]methyl]sulfinyl]-1H-benzimidazole (dexlansoprazole), a compound that inhibits gastric acid secretion, is marketed under trade name of KAPIDEX®.

U.S. Pat. No. 5,948,789 describes the preparation of R-isomer of lansoprazole by chiral oxidation of 2-[[[3-methyl-4-(2,2,2-trifluoroethoxy)-2-pyridinyl]methyl]thio]-1H-benzimidazole compound using titanium(IV) isopropoxide and (+)-diethyl L-tartrate and isolated as an oil. The obtained oil compound is chromatographed and treated with acetonitrile three times to achieve chiral purity.

U.S. Pat. No. 6,462,058 and U.S. Pat. No. 6,664,276 claim anhydrous crystal and 1.5 hydrate crystals of dexlansoprazole and also disclose the process for preparation of the claimed crystals form.

U.S. Pat. No. 6,002,011 describes the preparation of racemic lansoprazole monohydrate, monoethanol solvate by using mixture of ethanol-water mixture.

WO9602535 and WO9702261 describe the isolation of dexlansoprazole with an optical purity of 99.6% e.e.

WO2008077866 describes the preparation of lansoprazole hydrate acetonitrile solvate by dissolving lansoprazole in a mixture of acetonitrile and water and isolated as solvate.

WO2009088857 discloses various solvates crystals of dexlansoprazole, preferably methanol solvate, ethanol solvate and its hydrate, and isopropanol solvate and its hydrate crystals.

WO2010039885 discloses various crystal forms of dexlansoprazole such as, form X, form XI, form XII, form XIII, form XIV and processes for preparation. U.S. Pat. No. 5,578,732 (the '732 patent) discloses the 2-[[[3-methyl-4-(2,2,2-trifluoroethoxy)-2-pyridinyl]methyl]thio]-1H-benzimidazole monohydrate, and its use in the preparation of lansoprazole.

U.S. Pat. No. 6,002,011 (the '011 patent) discloses the process for preparation of 2-[[[3-methyl-4-(2,2,2-trifluoroethoxy)-2-pyridinyl]methyl]thio]-1H-benzimidazole monohydrate crystal compound.

WO2006074952A1 patent discloses the 2-[[[3-Methyl-4-(2,2,2-trifluoroethoxy)-2-pyridinyl]methyl]thio]-1H-benzimidazole 2-propanol solvate.

Considering the commercial importance of dexlansoprazole, applicant focused developing a process for dexlansoprazole with an enhanced chiral purity and reduced impurity. Applicant surprisingly found that the isolation of dexlansoprazole as diol solvate enhances the chiral purity and reduce the sulphone impurity. The inventors of the present invention conducted an intensive investigation directed to improvements in the above-mentioned aspects for the purpose of providing substantially pure dexlansoprazole compound from the diol solvate of dexlansoprazole.

### OBJECTIVES OF THE INVENTION

An objective of the present invention is to provide (R)-2-[[[3-methyl-4-(2,2,2-trifluoroethoxy)-2-pyridinyl]methyl]sulfinyl]-1H-benzimidazole diol solvate and its hydrate such as propylene glycol, chloropropylene glycol, butanediol, ethylene glycol and the like, with good stability, purity and yield.

Yet another objective of the present invention is to provide a process for the preparation of diol solvate of (R)-2-[[[3-methyl-4-(2,2,2-trifluoroethoxy)-2-pyridinyl]methyl]sulfinyl]-1H-benzimidazole.

Still another objective of the present invention is to provide the process for preparation of pure dexlansoprazole, from diol solvates and of dexlansoprazole, with good chiral purity and stability.

Still yet another objective of the present invention is to provide the anhydrous form of 2-[[[3-methyl-4-(2,2,2-trifluoroethoxy)-2-pyridinyl]methyl]thio]-1H-benzimidazole and also provides the process for preparation of anhydrous form.

### SUMMARY OF THE INVENTION

Accordingly, a first aspect of the present invention is directed to (R)-2-[[[3-methyl-4-(2,2,2-trifluoroethoxy)-2-pyridinyl]methyl]sulfinyl]-1H-benzimidazole diol solvate and its hydrate.

The second aspect of the present invention is to provide (R)-2-[[[3-methyl-4-(2,2,2-trifluoroethoxy)-2-pyridinyl]methyl]sulfinyl]-1H-benzimidazole ethylene glycol solvate.

The third aspect of the present invention is to provide (R)-2-[[[3-methyl-4-(2,2,2-trifluoroethoxy)-2-pyridinyl]methyl]sulfinyl]-1H-benzimidazole propylene glycol solvate.

The fourth aspect of the present invention is to provide (R)-2-[[[3-methyl-4-(2,2,2-trifluoroethoxy)-2-pyridinyl]methyl]sulfinyl]-1H-benzimidazole chloropropylene glycol solvate.

The fifth aspect of the present invention is to provide (R)-2-[[[3-methyl-4-(2,2,2-trifluoroethoxy)-2-pyridinyl]methyl]sulfinyl]-1H-benzimidazole 1,2-butanediol solvate.

The sixth aspect of the present invention is to provide (R)-2-[[[3-methyl-4-(2,2,2-trifluoroethoxy)-2-pyridinyl]methyl]sulfinyl]-1H-benzimidazole 1,4-butanediol solvate.

The seventh aspect of the present invention is directed to a process for preparing (R)-2-[[[3-methyl-4-(2,2,2-trifluoroethoxy)-2-pyridinyl]methyl]sulfinyl]-1H-benzimidazole diol solvate, comprising the steps of:
i) treating dexlansoprazole with diol selected from group consisting of 1,2-propylene glycol, 1,3-propylene glycol, chloropropylene glycol, ethylene glycol, 1,2-butanediol or 1,4-butanediol in an solvent;
ii) optionally heating the reaction mass; and
iii) isolating dexlansoprazole diol solvate.

The eighth aspect of the present invention is directed to process for preparation of pharmaceutically acceptable dexlansoprazole from dexlansoprazole diol solvate, comprising the steps of treating dexlansoprazole diol solvate with water.

In ninth aspect of the present invention is directed to anhydrous form of 2-[[[3-methyl-4-(2,2,2-trifluoroethoxy)-2-pyridinyl]methyl]thio]-1H-benzimidazole of formula (II).

In tenth aspect of the present invention directed to a process for preparing anhydrous form of 2-[[[3-methyl-4-(2,2,2-trifluoroethoxy)-2-pyridinyl]methyl]thio]-1H-benzimidazole of formula (II) which comprising the steps of:
i) refluxing 2-[[[3-methyl-4-(2,2,2-trifluoroethoxy)-2-pyridinyl]methyl]thio]-1H-benzimidazole or its hydrate in an organic solvent under azeotropic condition;
ii) cooling the solution; and
iii) isolating the anhydrous form of 2-[[[3-methyl-4-(2,2,2-trifluoroethoxy)-2-pyridinyl]methyl]thio]-1H-benzimidazole.

### BRIEF DESCRIPTION OF DRAWINGS

FIG. 1 illustrates X-ray powder diffraction pattern of crystalline form of (R)-2-[[[3-methyl-4-(2,2,2-trifluoroethoxy)-2-pyridinyl]methyl]sulfinyl]-1H-benzimidazole propylene glycol solvate according to the present invention.

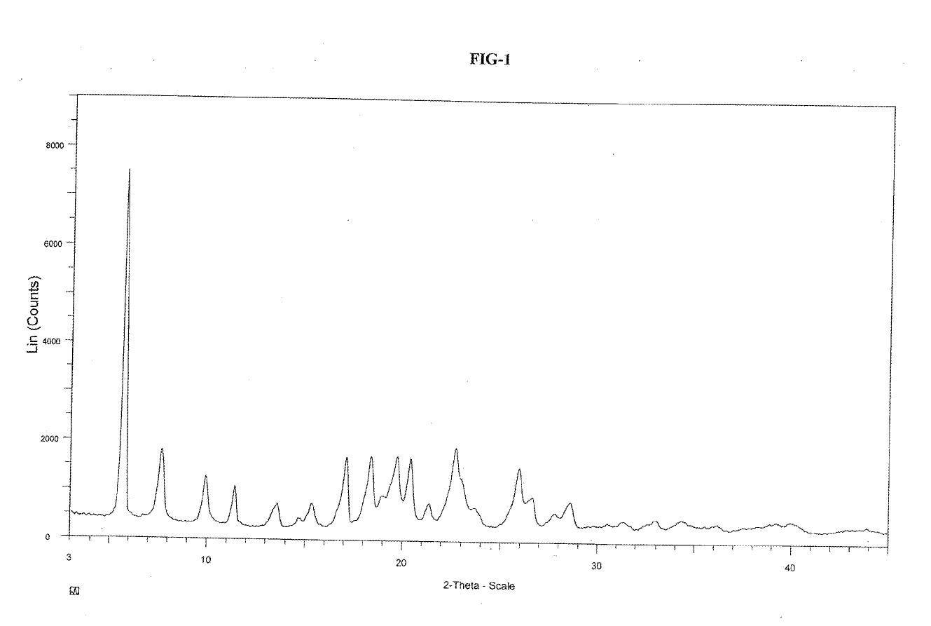

FIG. 2 illustrates X-ray powder diffraction pattern of crystalline form of (R)-2-[[[3-methyl-4-(2,2,2-trifluoroethoxy)-2-pyridinyl]methyl]sulfinyl]-1H-benzimidazole chloropropylene glycol solvate according to the present invention.

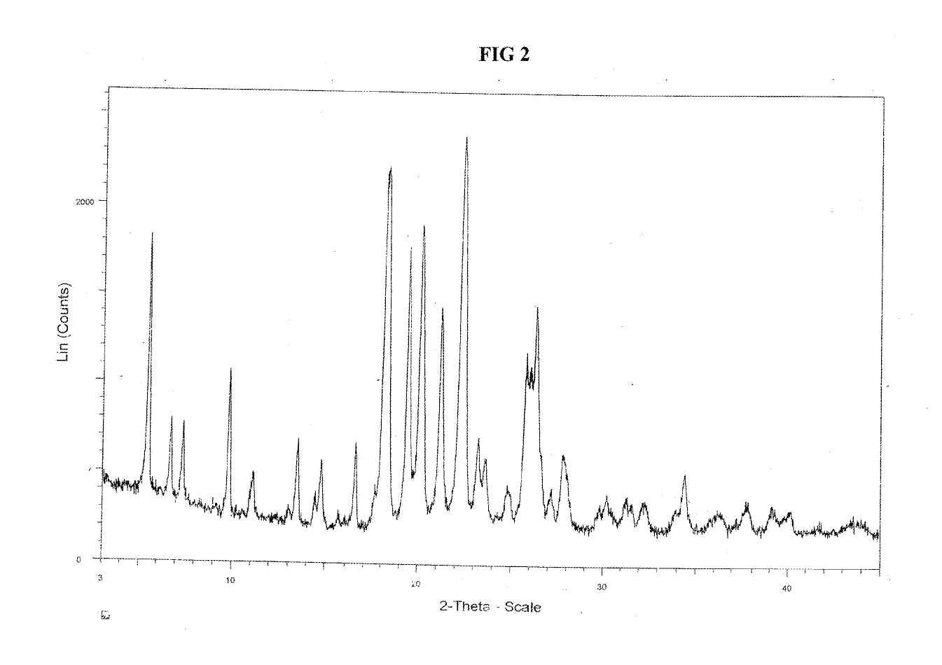

FIG. 3 illustrates X-ray powder diffraction pattern of crystalline form of (R)-2-[[[3-methyl-4-(2,2,2-trifluoroethoxy)-2-pyridinyl]methyl]sulfinyl]-1H-benzimidazole 1,2-butanediol solvate according to the present invention.

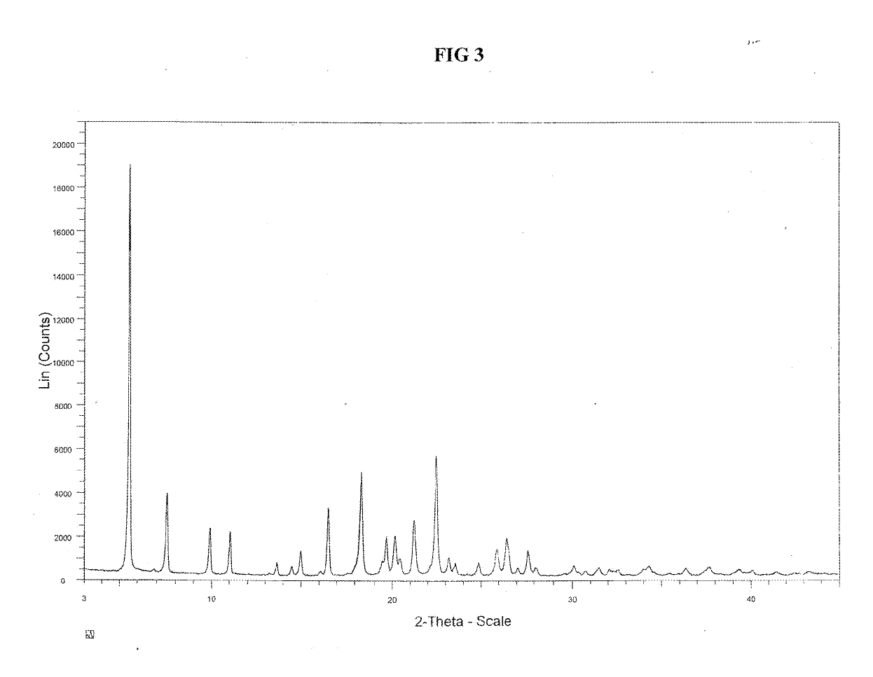

FIG. 4 illustrates X-ray powder diffraction pattern of crystalline form of (R)-2-[[[3-methyl-4-(2,2,2-trifluoroethoxy)-2-pyridinyl]methyl]sulfinyl]-1H-benzimidazole 1,4-butanediol solvate according to the present invention.

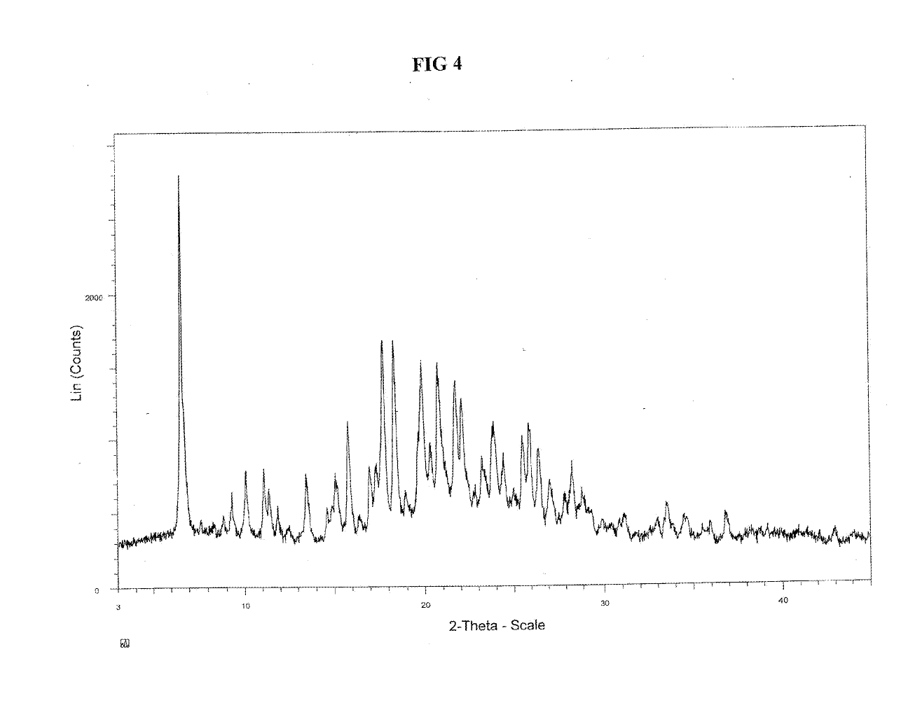

FIG. 5 shows X-ray powder diffraction pattern of anhydrous form of 2-[[[3-methyl-4-(2,2,2-trifluoroethoxy)-2-pyridinyl]methyl]thio]-1H-benzimidazole of formula (II) according to the present invention

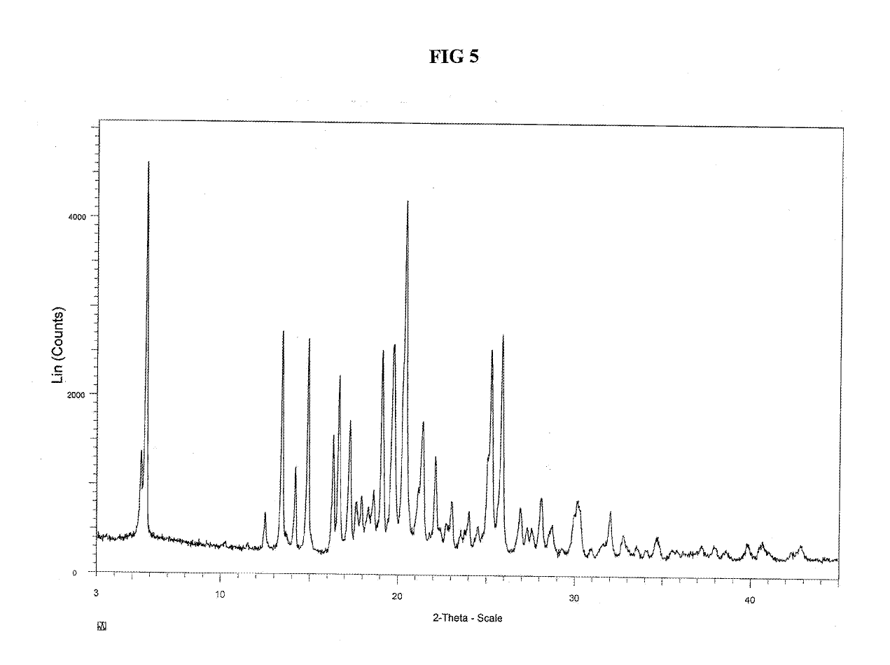

The PXRD was obtained under the following conditions:

| | |
|---|---|
| Make | BRUKER AXS |
| Model | D8 ADVANCE |
| Data handling system | EVA 12.0.0.0. |
| ANODE | COPPER |
| RADIATION | COPPER K alpha-1 |
| WAVELENGTH | 1.5406° |
| CURRENT &VOLTAGE | 40 kV 30 mA |

### DETAILED DESCRIPTION OF THE INVENTION

In a first embodiment of the present invention, (R)-2-[[[3-methyl-4-(2,2,2-trifluoroethoxy)-2-pyridinyl]methyl]sulfinyl]-1H-benzimidazole diol solvate according to the present invention can be isolated either as an amorphous form or in crystalline form, preferably in crystalline form. The solvate according to the present invention can be isolated either in anhydrous form or its hydrated form. The dexlansoprazole solvate according to the present invention includes hemi-, semi-, mono-, sesqui-, di-, tri-, tetra-, penta-, etc. The term “hydrated” refers to a combination of water with a “dexlansoprazole diol solvate” wherein the water retains its molecular state as water and is either absorbed, adsorbed or contained within a crystal lattice of the a dexlansoprazole diol solvate. Accordingly the term dexlansoprazole diol solvate referred in this specification includes its hydrate, which includes hemi-, semi-, mono-, sesqui-, di-, tri-, tetra-, penta-, etc.

Applicant surprisingly found that the isolation of dexlansoprazole diol solvate enhances the chiral purity of the dexlansoprazole along with reduction of sulphone impurity. The isolated solvate has high storage stability and purity. The conventional process of isolation of dexlansoprazole yields the product with less chiral purity and higher sulphone content, requiring tedious purification process. Surprisingly, the isolation as solvate of the present invention overcomes the limitations associated with conventional process, yielding dexlansoprazole with high purity and yield. None of the literature per se discloses the dexlansoprazole diol solvate, and constitutes novelty of the present invention.

The identification of suitable solvates requires thorough investigation of many factors, including but not limited to (i) stability of the solvates (ii) ease of isolation, (iii) yield and quality of dexlansoprazole that are prepared from these solvates (iii) color and degradation pattern of solvate and (iv) cost; apart from this the conventional preparation of dexlansoprazole require more purification, involving the use of column, to achieve chirality, which affects the quantity of final API. Considering the difficulty associated with the prior art process, the present invention provides (R)-2-[[[3-methyl-4-(2,2,2-trifluoroethoxy)-2-pyridinyl]methyl]sulfinyl]-1H-benzimidazole diol solvate is easy to implement in plant, associated with low cost of manufacture, and providing good yield and high purity including chiral purity. Accordingly the present invention provides dexlansoprazole 1,2-propylene glycol solvate, dexlansoprazole 1,3-propylene glycol solvate, dexlansoprazole chloropropylene glycol solvate, dexlansoprazole ethylene glycol solvate, dexlansoprazole 1,2-butanediol solvate or dexlansoprazole 1,4-butanediol solvate.

In a second embodiment of the present invention, crystalline R)-2-[[[3-methyl-4-(2,2,2-trifluoroethoxy)-2-pyridinyl]methyl]sulfinyl]-1H-benzimidazole propylene glycol solvate is characterized by a powder X-ray diffraction pattern with peaks at 5.70, 7.64, 9.90, 11.37, 13.52, 14.66, 15.32, 17.08, 18.37, 18.97, 19.70, 20.39, 21.35, 22.69, 23.02, 23.66, 25.94, 26.62, 27.80, 28.57 and 30.54±0.2 degrees 2θ.

In a third embodiment of the present invention, crystalline (R)-2-[[[3-methyl-4-(2,2,2-trifluoroethoxy)-2-pyridinyl]methyl]sulfinyl]-1H-benzimidazole chloro propylene glycol solvate is characterized by a powder X-ray diffraction pattern with peaks at 5.45, 6.68, 7.33, 9.79, 11.11, 13.45, 14.44, 14.77, 16.53, 17.62, 18.25, 19.44, 20.15, 21.22, 22.36, 23.23, 23.62, 24.80, 25.81, 26.32, 27.16, 27.83, 30.21, 31.31, 31.62, 32.41 and 34.44±0.2 degrees 2θ.

In a fourth embodiment of the present invention, crystalline (R)-2-[[[3-methyl-4-(2,2,2-trifluoroethoxy)-2-pyridinyl]methyl]sulfinyl]-1H-benzimidazole 1,2-butanediol solvate is characterized by a powder X-ray diffraction pattern with peaks at 5.53, 7.49, 9.88, 11.02, 13.61, 14.46, 14.95, 16.05, 16.53, 18.30, 19.67, 20.42, 21.24, 22.49, 24.88, 26.47, 27.10 and 27.70±0.2 degrees 2θ. The isolated solvate contain about 15% to 40% of 1.2-butanediol and moisture content in the range of 0.5-10%, preferably 2-10%.

In a fifth embodiment of the present invention, crystalline (R)-2-[[[3-methyl-4-(2,2,2-trifluoroethoxy)-2-pyridinyl]methyl]sulfinyl]-1H-benzimidazole 1,4-butanediol solvate is characterized by a powder X-ray diffraction pattern with peaks at 6.61, 7.60, 8.29, 8.79, 9.26, 10.06, 11.09, 11.36, 11.85, 12.44, 13.47, 14.60, 15.08, 15.79, 16.38, 16.98, 17.35, 17.75, 18.36, 18.93, 19.88, 20.34, 20.80, 21.74, 22.10, 22.82, 23.25, 23.46, 23.89, 24.42, 25.00, 25.57, 25.94, 26.46, 27.11, 27.98, 28.41, 28.98, 29.33, 29.90 and 30.44±0.2 degrees 2θ.

In a sixth embodiment of the present invention, treating dexlansoprazole with diol as mentioned in step (i) includes (a) mixing dexlansoprazole and diol in the presence or absence of solvent (b) obtaining solution of dexlansoprazole in a solvent followed by addition of diol (iii) preparing slurry of dexlansoprazole in a solvent followed by addition of diol. The reaction mass thus obtained was optionally heated to a temperature in the range of 25° C. to reflux temperature of the solvent used. To reaction mass organic base selected from group comprising triethylamine, N,N-diisopropylethylamine, Diazabicycloundecene (DBU), pyridine, TMG, N,N-dimethylamine, N,N-diisopropylamine, and the like was optionally added.

In a seventh embodiment of the present invention, the solution of dexlansoprazole as represented above in point (b) is obtained either directly from the reaction, for e.g. chiral oxidation of 2-[[[3-methyl-4-(2,2,2-trifluoroethoxy)-2-pyridinyl]methyl]thio]-1H-benzimidazole, or by dissolving crude dexlansoprazole in a solvent. To the clear solution, the diol such as 1,2-propylene glycol, 1,3-propylene glycol, chloropropylene glycol, ethylene glycol, 1,2-butanediol or 1,4-butanediol was added.

In a eighth embodiment of the present invention, the solvent used in step (i) in the preparation of dexlansoprazole diol solvate is selected from group comprising toluene, xylene, isopropyl acetate, n-butyl acetate tert-butyl acetate, methyl tert-butyl ether, diisopropyl ether, ethyl acetate, dichloromethane, tetrahydrofuran, dimethylformamide, dimethylacetamide, hexane, heptane, acetone, methyl isobutyl ketone, water and the like or mixture thereof.

In a ninth embodiment of the present invention, the isolation of dexlansoprazole diol solvate is done by cooling the solution or by adding anti-solvent.

In a tenth embodiment of the present invention the dexlansoprazole diol solvate thus obtained was converted to dexlansoprazole by treating the solution of dexlansoprazole diol solvate in water and in the presence or absence of water immiscible solvent such as dichloromethane, ethyl acetate, toluene, methyl tert-butyl ether, xylene, isopropyl acetate, tert-butyl acetate, and the like. The pH during extraction is maintained about 6.5 to 9.0. The extracted dexlansoprazole in water immiscible solvent was isolated by using the conventional methods disclosed in the examples or by distilling the solvent at neutral pH followed by addition of anti-solvent, preferably pre-heated anti-solvent, or vice-versa.

In a eleventh embodiment of the present invention, the present invention provides anhydrous form of 2-[[[3-methyl-4-(2,2,2-trifluoroethoxy)-2-pyridinyl]methyl]thio]-1H-benzimidazole, which is a starting material in the preparation of dexlansoprazole. The isolation of 2-[[[3-methyl-4-(2,2,2-trifluoroethoxy)-2-pyridinyl]methyl]thio]-1H-benzimidazole as anhydrous form is highly useful in the asymmetric oxidation. It should be noted that the presence of moisture during asymmetric oxidation affects the completion of reaction thereby yield is low. The water was separated from the reaction mass by conventional method or by using dean-stark apparatus. After removal of water, which is confirmed by checking the moisture content of the reaction mass, the reaction mass was cooled to a temperature in the range of (−) 10° C. to 10° C.; most preferably at a temperature in the range of 0° C. to 5° C. and isolated as anhydrous form. The moisture content of 2-[[[3-methyl-4-(2,2,2-trifluoroethoxy)-2-pyridinyl]methyl]thio]-1H-benzimidazole compound is determined by thermogravimetric analysis or by Karl Fischer and found to be less than 0.15%. Further the organic solvent used for preparation of anhydrous form of 2-[[[3-methyl-4-(2,2,2-trifluoroethoxy)-2-pyridinyl]methyl]thio]-1H-benzimidazole in step (i) is selected from group comprising toluene, benzene, xylene, sulfolane, ethyl acetate and the like; preferably toluene. The anhydrous compound of formula (II) is converted to dexlansoprazole by the method known in prior art or by following the procedure disclosed in Reference example-1.

Many other beneficial results can be obtained by applying disclosed invention in a different manner or by modifying the invention with the scope of disclosure. The method disclosed in this application can be extended to other prazole derivative for enhancing chiral purity.

The present invention is provided by the examples below, which are provided by way of illustration only and should not be considered to limit the scope of the invention.

### Example 1

### Preparation of (R)-2-[[[3-methyl-4-(2,2,2-trifluoroethoxy)-2-pyridinyl]methyl]sulfinyl]-1H-benzimidazole 1,2-propylene glycol solvate of Formula (I)

To a solution of crude (R)-2-[[[3-methyl-4-(2,2,2-trifluoroethoxy)-2-pyridinyl]methyl]sulfinyl]-1H-benzimidazole in toluene (or IPE) was added 1,2-propylene glycol (propane-1,2-diol) and few drops of triethylamine. The reaction mixture was heated to 25-45° C. and gradually cooled to 5-10° C. The obtained compound was filtered and washed with toluene.

HPLC purity: >99.40%; Sulfone content: <0.30%; Chiral HPLC purity: 99.15-99.80%.

### Example 2

### Preparation of (R)-2-[[[3-methyl-4-(2,2,2-trifluoroethoxy)-2-pyridinyl]methyl]sulfinyl]-1H-benzimidazole propylene glycol solvate of Formula (I)

To a solution of crude (R)-2-[[[3-methyl-4-(2,2,2-trifluoroethoxy)-2-pyridinyl]methyl]sulfinyl]-1H-benzimidazole in tert-butyl acetate at 50-60° C. was added 1,2-propylene glycol. The reaction mixture was maintained for 1-2 hours at 0-5° C. The obtained compound was filtered, washed with chilled tert-butyl acetate and dried under vacuum at 45° C.

HPLC purity: 99.72%; Sulfone content: 0.19%; Chiral HPLC purity: 99.94%.

### Example 3

### Preparation of dexlansoprazole from its propylene glycol solvate

(R)-2-[[[3-methyl-4-(2,2,2-trifluoroethoxy)-2-pyridinyl]methyl]sulfinyl]-1H-benzimidazole propylene glycol solvate was dissolved in dichloromethane, washed with water and brine solution. Organic layer was dried over sodium sulphate and then filtered. Carbon was added to dichloromethane layer, stirred for 15 minutes, and filtered through hyflo bed. Partial amount of dichloromethane was removed by distillation. Diisopropyl ether was taken separately and heated to 25-30° C. MDC solution was added to hot diisopropyl ether and stirred for 1 hour at 25-30° C. The obtained product was filtered, washed with diisopropyl ether and dried under vacuum at 45° C. for 12-15 hrs.

HPLC purity: 99.80%; Sulfone content: 0.20%; Chiral HPLC purity: 99.94%.

### Example 4

### Preparation of (R)-2-[[[3-methyl-4-(2,2,2-trifluoroethoxy)-2-pyridinyl]methyl]sulfinyl]-1H-benzimidazole chloro propylene glycol solvate of Formula I)

To a solution of crude (R)-2-[[[3-methyl-4-(2,2,2-trifluoroethoxy)-2-pyridinyl]methyl]sulfinyl]-1H-benzimidazole in tert-butyl acetate was added to chloropropylene glycol. The reaction mixture was heated to 50-60° C. and gradually cooled to 5-10° C. The obtained compound was filtered and washed with tert-butyl acetate.

Sulfone content: 0.26%; Chiral HPLC purity: 99.90% (e,e).

### Example 5

### Preparation of (R)-2-[[[3-methyl-4-(2,2,2-trifluoroethoxy)-2-pyridinyl]methyl]sulfinyl]-1H-benzimidazole 1,2-butanediol solvate of Formula (I)

To a solution of crude (R)-2-[[[3-methyl-4-(2,2,2-trifluoroethoxy)-2-pyridinyl]methyl]sulfinyl]-1H-benzimidazole in tert-butyl acetate was added to 1,2-butanediol. The reaction mixture was heated to 50-60° C. and gradually cooled to 5-10° C. The obtained compound was filtered and washed with tert-butyl acetate.

Chiral HPLC purity: 99.80% (e,e);

### Example 6

### Preparation of (R)-2-[[[3-methyl-4-(2,2,2-trifluoroethoxy)-2-pyridinyl]methyl]sulfinyl]-1H-benzimidazole 1,4-butanediol solvate of Formula (I)

To a solution of crude (R)-2-[[[3-methyl-4-(2,2,2-trifluoroethoxy)-2-pyridinyl]methyl]sulfinyl]-1H-benzimidazole in toluene was added to 1,4-butanediol. The reaction mixture was heated to 50-60° C. and gradually cooled to 5-10° C. The obtained compound was filtered and washed with toluene.

Chiral HPLC purity: 99.68% (e,e)

### Example 7

### Preparation of (R)-2-[[[3-methyl-4-(2,2,2-trifluoroethoxy)-2-pyridinyl]methyl]sulfinyl]-1H-benzimidazole 1,2-butanediol solvate of Formula (I)

To a solution of crude (R)-2-[[[3-methyl-4-(2,2,2-trifluoroethoxy)-2-pyridinyl]methyl]sulfinyl]-1H-benzimidazole in toluene (obtained directly by dissolving the residue in reference example-1 using toluene, wherein 50 g of 2-[[[3-methyl-4-(2,2,2-trifluoroethoxy)-2-pyridinyl]-methyl]thio]-1H-benzimidazole was used) was added to 1,2-butanediol (50 mL) in diisopropyl ether. The reaction mixture was heated to 45-50° C. and gradually cooled to 25-35° C. and diisopropyl ether was added over it under stirring, followed by addition of water. The reaction mass further cooled to 0-5° C. The obtained compound was filtered and washed with diisopropyl ether. The obtained butanediol solvate compound was treated with toluene and heated to 40-45° C. and filtered to remove undissolved material. Then the filtrate was gradually cooled to 25-35° C., optionally addition of butanediol and water. The reaction mass was maintained for 1-2 hours, filtered and washed with toluene.

Chiral purity: 100.00%; Purity by HPLC: 99.82%; Sulfone: 0.18

### Example 8

### Preparation of (R)-2-[[[3-methyl-4-(2,2,2-trifluoroethoxy)-2-pyridinyl]methyl]sulfinyl]-1H-benzimidazole 1,4-butanediol solvate of Formula (I)

To a solution of crude (R)-2-[[[3-methyl-4-(2,2,2-trifluoroethoxy)-2-pyridinyl]methyl]sulfinyl]-1H-benzimidazole in toluene was added to 1,4-butanediol in diisopropyl ether. The reaction mixture was heated to 45-50° C. and gradually cooled to 25-35° C. and diisopropyl ether was added over it under stirring. The reaction mass was further cooled to 0-5° C. The obtained compound was filtered and washed with diisopropyl ether. The obtained butanediol solvate compound was treated with toluene and heated to 40-45° C. and filtered to remove undissolved material. To filtrate, butanediol and water was added and cooled to 25-35° C. The reaction mass was maintained for 1-2 hours, filtered and washed with toluene.

### Yield: 55% w/w

### Example 9

### Preparation of dexlansoprazole

(R)-2-[[[3-methyl-4-(2,2,2-trifluoroethoxy)-2-pyridinyl]methyl]sulfinyl]-1H-benzimidazole 1, 2 or 1,4-butanediol solvate was treated with dichloromethane and water, and pH of the solution adjusted to 7.5-9 using triethylamine under stirred condition. The organic layer separated, washed with brine solution and partially distilled under vacuum at the same pH by addition of base. To residue acetone was added, the acetone layer was subjected to carbon treatment and distilled up to 1-2 volume. The obtained reaction solution was slowly added to preheated diisopropyl ether (40-45° C.) or vice-versa and cooled to 25-30° C. The obtained product filtered, washed with diisopropyl ether and dried under vacuum.

HPLC Purity: 99.75%; Chiral purity: 99.88% (R-Isomer); Sulfone content: 0.20%

### Example 10

### Preparation of anhydrous 2[[[3-methyl-4-(2,2,2-trifluoroethoxy)-2-pyridinyl]methyl]thio]-1H-benzimidazole of Formula (II)

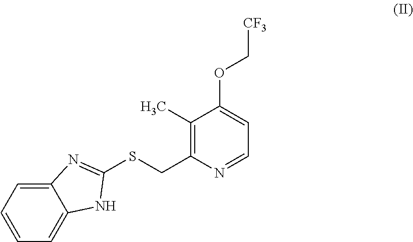 [SMILES: Cc1c(OCC(F)(F)F)ccnc1CSc1nc2ccccc2[nH]1]

To a stirred suspension of (2-chloromethyl)-3-methyl-4-(2,2,2-trifluoroethoxy)pyridine hydrochloride (250 g) and sodium carbonate in methanol, 2-mercapto-benzimidazole (142 g) was added and heated to reflux temperature. After completion of the reaction, reaction mass was cooled, DM water was added over to the reaction mixture under stirring. The obtained 2-[[[3-methyl-4-(2,2,2-trifluoroethoxy)-2-pyridinyl]methyl]thio]-1H-benzimidazole was filtered out from the solution mixture under vacuum and washed with water. The obtained wet product was taken in toluene and heated to reflux under azeotropic condition to remove water. After complete removal of moisture, cooled to 0-5° C. and the solid obtained was filtered and dried under vacuum at 85-90° C. for 12 hours. The isolated product contains less than 0.15% moisture content.

Dry weight: 255-280 g; Yield: 80-88%; HPLC Purity: NLT 99%

### Reference Example (1)

### Preparation of crude (R)-2-[[[3-methyl-4-(2,2,2-trifluoroethoxy)-2-pyridinyl]methyl]sulfinyl]-1H-benzimidazole (Crude R-lansoprazole) of Formula (I)

2-[[[3-methyl-4-(2,2,2-trifluoroethoxy)-2-pyridinyl]-methyl]thio]-1H-benzimidazole was dissolved in toluene. (+)-Diethyl tartrate was added and heated to 55-60° C. To the solution was added titanium(IV)isopropoxide and stirred for 1 hour at 55-60° C. Reaction mixture cooled to 25-30° C. and diisopropylethylamine was added over it. To the solution was added cumene hydroperoxide in toluene at 0 to −3° C. Reaction mixture maintained for 3 hours. After completion of reaction, reaction mixture was quenched with 20% N,N dimethylamine solution followed by separation of aqueous layer. The separated aqueous was washed with dichloromethane. The pH of the aqueous layer was adjusted to 9.0-9.2 using acetic acid and extracted into toluene. Organic layer was concentrated to a residue. To the residue, dichloromethane was added over it to dissolve the gummy material followed by addition of isopropyl ether. After stirring for 2 hours, the product was filtered and dried under vacuum at 45° C. for 12 hours. (Alternatively the gummy material was dissolved in toluene/IPE followed to addition of diol reagent and optionally water to yield diol solvate of dexlansoprazole)

HPLC purity: 99.00%; Sulphone content: 0.84%; Chiral purity: ˜97% (e,e)

### Comparison of Various Solvate Purity

| | | | CHIRAL |
|---|---|---|---|
| | PURITY | SULPHONE | PURITY |
| Starting material | 99.90% | — | — |
| (Thio compound) | | | |
| Crude | ~97.00-99.00% | ~1.00-2.00% | 89.00-97.00% |
| dexlansoprazole | | | |
| Dexlansoprazole | 99.40-99.72% | 0.19-0.30% | 99.15-99.94% |
| propylene glycol | | | |
| solvate | | | |
| Dexlansoprazole | >99.5% | 0.26-0.30% | 99.00% |
| chloropropylene | | | |
| glycol solvate | | | |
| Dexlansoprazole | 99.50-99.90% | 0.05-0.35% | 99.5-99.99% |
| 1,2-butanediol | | | |
| solvate | | | |

The above table clearly indicates that the isolation of dexlansoprazole as its diol solvate and its hydrate enhances the chiral purity and provides simple process that avoids chromatographic purification to achieve the chiral purity.

1-23. (canceled)

24. A Butanediol solvate of Dexlansoprazole and its hydrate.

25. The butanediol solvate of dexlansoprazole as claimed in claim 24 is selected from 1,2-butanediol solvate of dexlansoprazole and 1,4-butanediol solvate of dexlansoprazole.

26. A process for the preparation of butanediol solvate of dexlansoprazole as claimed in claim 24 comprises of
i) treating dexlansoprazole with 1,2-butanediol or 1,4-butanediol in an solvent;
ii) optionally heating the reaction mass; and
iii) isolating dexlansoprazole butanediol solvate.

27. The process according to claim 26, wherein the solvent used in step (i) is selected from toluene, xylene, isopropyl acetate, n-butyl acetate tert-butyl acetate, methyl tert-butyl ether, isopropyl ether, ethyl acetate dichloromethane, tetrahydrofuran, dimethylformamide, dimethylacetamide, hexane, heptane, acetone, methyl isobutyl ketone, water or mixture thereof.

28. The process according to claim 26, wherein reaction mass in step (ii) heated to 25° C. to reflux temperature of the solvent used.

29. The process according to claim 26, wherein isolation of dexlansoprazole butanediol solvate is done by cooling the solution or by adding anti-solvent.

30. An improved process for preparation of dexlansoprazole which comprises steps of:
i) treating dexlansoprazole with butanediol in a solvent;
ii) isolating dexlansoprazole butanediol solvate and its hydrate;
iii) treating the dexlansoprazole butanediol solvate with water; and
iv) isolating the dexlansoprazole.

31. The process according to claim 30, wherein the solvent used in step (i) is selected from the group comprising toluene, xylene, isopropyl acetate, n-butyl acetate tert-butyl acetate, methyl tert-butyl ether, isopropyl ether, ethyl acetate, dichloromethane, tetrahydrofuran, dimethylformamide, dimethylacetamide, hexane, heptane, acetone, methyl isobutyl ketone, water or mixture thereof.

32. The process according to claim 30, wherein the butanediol used in step (i) is selected from 1,2-butanediol and 1,4-butanediol.

33. The process according to claim 30, wherein the dexlansoprazole is isolated by extracting the reaction mass of step (iv) with water immiscible solvent, followed by distilling the reaction mass optionally in the presence of organic base; and addition of anti-solvent.

34. A 1,2-butanediol solvate of (R)-2-[[[3-methyl-4-(2,2,2-trifluoroethoxy)-2-pyridinyl]methyl]sulfinyl]-1H-benzimidazole having a powder X-ray diffraction pattern with peaks at 5.53, 7.49, 9.88, 11.02, 13.61, 14.46, 14.95, 16.05, 16.53, 18.30, 19.67, 20.42, 21.24, 22.49, 24.88, 26.47, 27.10 and 27.70±0.2 degrees 2θ.

35. A 1,4-butanediol solvate of (R)-2-[[[3-methyl-4-(2,2,2-trifluoroethoxy)-2-pyridinyl]methyl]sulfinyl]-1H-benzimidazole having a powder X-ray diffraction pattern with peaks at 6.61, 7.60, 8.29, 8.79, 9.26, 10.06, 11.09, 11.36, 11.85, 12.44, 13.47, 14.60, 15.08, 15.79, 16.38, 16.98, 17.35, 17.75, 18.36, 18.93, 19.88, 20.34, 20.80, 21.74, 22.10, 22.82, 23.25, 23.46, 23.89, 24.42, 25.00, 25.57, 25.94, 26.46, 27.11, 27.98, 28.41, 28.98, 29.33, 29.90 and 30.44±0.2 degrees 2θ.

36. A process for preparing dexlansoprazole comprising: reacting dexlansoprazole butanediol solvate and its hydrate according to claim 24 to obtain dexlansoprazole.

37. An Anhydrous form of 2-[[[3-methyl-4-(2,2,2-trifluoroethoxy)-2-pyridinyl]methyl]thio]-1H-benzimidazole of formula (II)

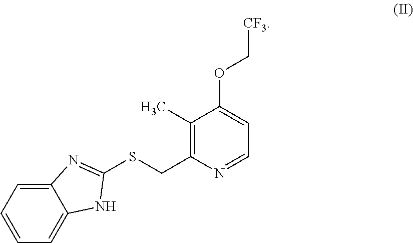 [SMILES: Cc1c(OCC(F)(F)F)ccnc1CSc1nc2ccccc2[nH]1]

38. A process for preparing anhydrous form of 2-[[[3-methyl-4-(2,2,2-trifluoroethoxy)-2-pyridinyl]methyl]thio]-1H-benzimidazole comprises refluxing 2[[[3-methyl-4-(2,2,2-trifluoroethoxy)-2-pyridinyl]methyl]thio]-1H-benzimidazole or its hydrate in an organic solvent under azeotropic condition till moisture content of the reaction mass reaches less than 0.5%.

39. The process as claimed in claim 38, wherein the organic solvent used is selected from group comprising toluene, benzene, xylene, sulfolane, ethyl acetate or mixture thereof.

40. A process for preparing dexlansoprazole or its solvate comprising: reacting an anhydrous form of 2-[[[3-methyl-4-(2,2,2-trifluoroethoxy)-2-pyridinyl]methyl]thio]-1H-benzimidazole to obtain dexlansoprazole or its solvate.
